(19) United States
(12) Patent Application Publication
(10) Pub. No.: US 2025/0032640 A1
(43) Pub. Date: Jan. 30, 2025

(54) TARGETED DRUG DELIVERY-RELEASE AND KILLING OF DRUG RESISTANT TUMORS AND PATHOGENS

(71) Applicant: RUBHU BIOLOGICS, INC., Athens, GA (US)

(72) Inventor: Surendra Jagannath CHAVAN, Lilburn, GA (US)

(21) Appl. No.: 18/709,362

(22) PCT Filed: Nov. 17, 2022
(86) PCT No.: PCT/US2022/080034

§ 371 Date: May 10, 2024

Related U.S. Application Data
(60) Provisional application No. 63/281,415, filed on Nov. 19, 2021.

(51) Int. Cl. A61K 47/69; A61K 47/42; C07K 16/08; C07K 16/10; C07K 16/20; C07K 16/30

(52) U.S. Cl. CPC A61K 47/6913; A61K 47/42; A61K 47/6901; C07K 16/082; C07K 16/10; C07K 16/1003; C07K 16/1063; C07K 16/205; C07K 16/30

(57) ABSTRACT

Disclosed herein is a method for controlled delivery of an agent in a subject that involves administering to the subject a biotinylated cell complex loaded with the agent; and then administering to the subject an effective amount of streptavidin/neutravidin crosslinking agent to crosslink the biotinylated complex. Also disclosed is a method for killing a drug resistant pathogen in a subject that involves administering to the subject biotinylated binding agents that specifically bind one or more antigens on the pathogen, and then administering to the subject an effective amount of streptavidin crosslinking agent to rupture the pathogen.

### CROSS-REFERENCE TO RELATED APPLICATIONS

This application claims benefit of U.S. Provisional Application No. 63/281,415, filed Nov. 19, 2021, which is hereby incorporated herein by reference in its entirety.

### SEQUENCE LISTING

This application contains a sequence listing filed in ST.26 format entitled “321813_2030_Sequence_Listing” created on Oct. 26, 2022. The content of the sequence listing is incorporated herein in its entirety.

### BACKGROUND

Antibiotic resistance leads to higher medical costs, prolonged hospital stays, and increased mortality. It is unlikely that traditional antibiotics will suffice as long-term solutions for the treatment of increasingly resistant bacterial infections. Nontraditional therapies, including monoclonal antibodies, deserve further attention and evaluation, as they are less likely to contribute to the increasing emergence of antibiotic resistance than antibiotics. There is a serious lack of treatment options for multidrug- and extensively drug-resistant M. tuberculosis and gram-negative pathogens, including Acinetobacter and Enterobacteriaceae (such as Klebsiella and E. coli) which can cause severe and often deadly infections that pose a particular threat in hospitals and nursing homes.

The evolution of drug resistance is facilitated by a number of factors, including increasing use of antibiotics and anti-parasitic drugs, the insufficient controls on prescription, inadequate compliance with treatment regimens, poor dosing, limited instruction to doctors in rural areas, lack of infection control, increasing frequency and speed of travel, which lead to the rapid spread of resistant organisms; and insufficient incentives for patients, physicians, or even governments to care about increasing resistance. It is important to distinguish between risk factors for the emergence of resistance (de novo resistance) and those for the spread of resistance (primary resistance).

WHO's new Global Antimicrobial Surveillance System (GLASS) reveals widespread occurrence of antibiotic resistance among 500,000 people with suspected bacterial infections across 22 countries. The world urgently needs to change the way it uses antibiotics. Even if new medicines are developed, without behavior change, antibiotic resistance will remain a major threat.

Majority of drug development strategy are focus on single pathway/single enzyme/single protein to limit toxicity and increase specificity. This safer approach leads in development of drug resistance over time.

### SUMMARY

Disclosed herein are compositions and methods to target multiple building block of membrane such as a surface proteins, lipids, or glycans (glycoprotein, glycolipid, or a proteoglycan), on the organism include but not limited to unicellular microorganism such as bacteria, virus, protozoa, procaryotic cells, also this strategy is applicable to multicellular organisms such as fungi, plant, and other animal cells/organs. Following are the key steps will be involved in the killing the organisms using this technology: 1) Identification of 2 or more surface specific proteins, glycoproteins or carbohydrates or polysaccharides or lipids or lipid moieties, which are abundantly and specifically express on the surface (Surface proteins or moieties with longer surface turn-around time will be better-one with longer surface presence); 2) Identification or development of Binding Agent (BA) such as antibodies or Fab fragment, or proteins which can bind to organism surface proteins (viz., nanomers, PNA, peptide etc) against the target protein(s) expressed on the surface of organism; 3) Biotinylation of binding agents (BA) or conjugation with bet entity/moiety; 4) loading/injecting these biotinylated (bet conjugated) binding agents; 5) Allow binding or biotinylated binding agent to targets and allow period to wash of the unbound or free binding agents; 6) Subsequent loading/injection of avidin/streptavidin/neutravidin (or multimeric anti-bet agent). This sequence of process will result in induction of physical strain on the membrane integrity to induce the rupture in the membrane wall resulting in pores in the membrane and killing of the organism. The selection of multiple surface proteins has two main advantages-1) chance of presence of all the selected anti-organism specific proteins on host tissues/organism will be rare and thus the no to less destruction of host organism; 2) targeted organism will have limited chances to develop the resistance to multiple proteins simultaneously and thus rare chances of development of drug resistance or it will take longer time.

Therefore, also disclosed herein is a method for controlled delivery and release of an agent in a subject that involves administering to the subject a biotinylated cell complex loaded with the agent; and then subsequently administering to the subject an effective amount of streptavidin/neutravidin crosslinking agent to crosslink the biotinylated complex. In some embodiments, the biotinylated cell complex is a non-nucleated or enucleated cell. For example, the cell can be a red blood cell (RBC) or platelet.

In some embodiments, the biotinylated complex is coated with one or more binding agents for targeted delivery. Examples of binding agents include antibodies, ligands, receptors, aptamers, DNA, RNA, nucleic acids and PNA. Other suitable binding agents suitable for targeted delivery in vivo can also be adapted for use in the disclosed methods.

In some embodiments, the binding agent specifically binds a tumor antigen. In these embodiments, the agent can be, for example, costimulatory molecules, death receptors, chemokines, and/or cytokines configured to kill cancer cells.

Also disclosed is a method for killing a drug resistant pathogen in a subject that involves administering to the subject biotinylated binding agents that specifically bind one or more antigens on the pathogen; and then administering to the subject an effective amount of streptavidin crosslinking agent to rupture the pathogen.

Again, examples of suitable binding agents include antibodies, ligands, receptors, aptamers, DNA, RNA, nucleic acids and PNA. Other suitable binding agents suitable for targeted delivery in vivo can also be adapted for use in the disclosed methods.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows RBC/Platelets/Exosomes/Liposomes loaded or conjugated with drug entities and will be subsequently biotinylated. These biotinylated target specific drug vehicles (TSV) will be conjugated with target specific-proteins (such as—antibodies, aptamers, peptides etc.) and/or target specific-PNA and/or -nucleic acids. These TSVs will deliver signal(s) (death/activation/proliferation/cell cycle arrest/differentiation). Once these TSVs accumulated in the target sites they will be subsequently treated with streptavidin (or similar entity) to the biotins present on TSVs. The streptavidin interaction with biotin will result in the physical strain on membrane of TSVs to release the drug entity in burst.

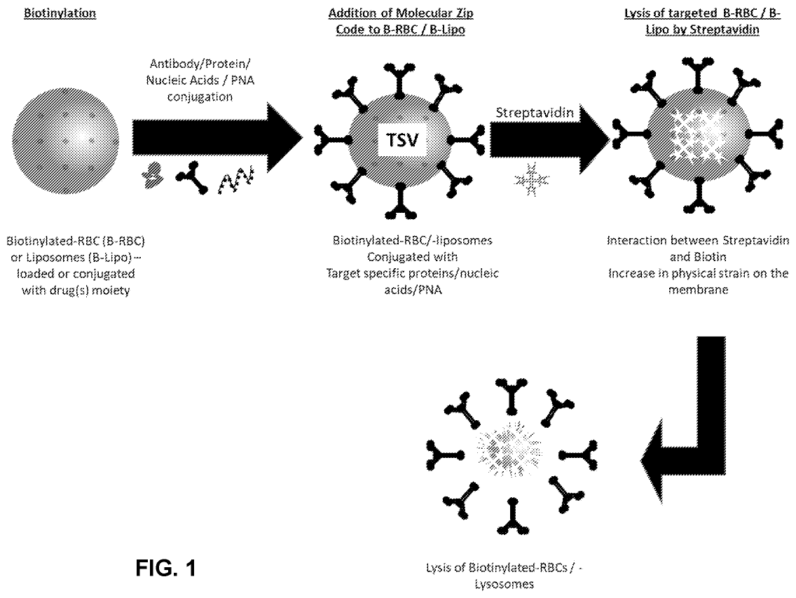

FIG. 2 shows target specific killing of drug resistant cancer cells or pathogens (such as bacteria, virus, fungi, yeast etc.) using multipronged approach to avoid development of resistant strains. For this strategy multiple entities viz, proteins, lipids, carbohydrates, glycans which have slow turnover rate expressed on the surface of target will be identified. The moieties (viz., antibodies, peptide, complementary glycans, PNA or ligands etc.) binding to these targets will be biotinylated or will be conjugated with complementary DNA or peptide-nucleic acids (PNA). These modified moieties will be allow to bind to targets and then will be subsequently treated with streptavidin or similar agents. The streptavidin interaction with biotin will result in the physical strain on membrane of cancer cells or pathogen to induce physical burst.

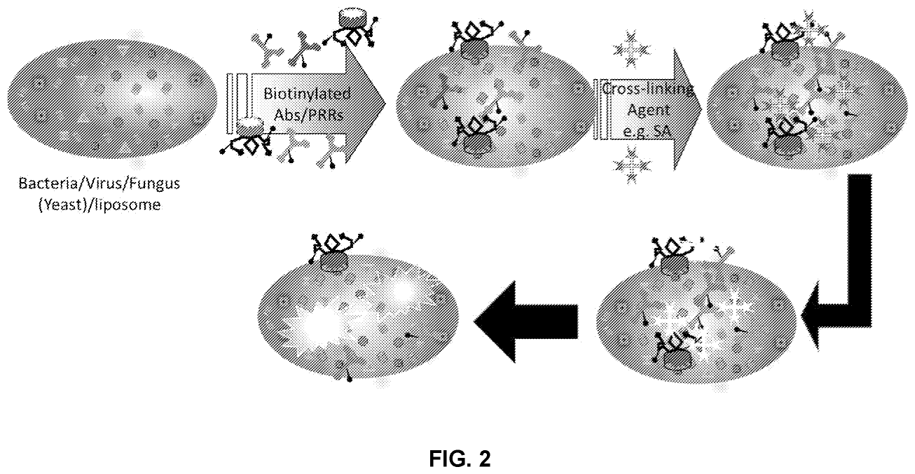

FIG. 3 shows target lysis of B-RBCs using Streptavidin-Biotin chemistry. RBCs were biotinylated using different amine-reactive biotinylation agents and then subjected to streptavidin over 30 hrs. Cond-A-Sulfo-NHS-Biotin; Cond-B-Sulfo-NHS-SS-Biotin; Cond-C-Sulfo-NHS-LC-Biotin; Cond-D-Sulfo-NHS-LC-LC-Biotin and Cond-E-Sulfo-NHS-PEG4-Biotin. Total # of intact RBC at different time were counted using hematocytometer and represented on Y-axis as percent of RBC lysis in comparison to control condition (the biotinylated RBC's were not treated with Streptavidin). Percent of RBC lysis at 18 hrs (gray) and 30 hrs (black). *˜ 50% RBC lyse in control condition at 30 hrs post streptavidin treatment was observed in sulfo-NHS-LC-LC-biotinylated RBCs.

FIG. 4 shows target lysis of B-RBCs using Streptavidin-Biotin chemistry. RBCs were biotinylated using different amine-reactive biotinylation agents and then subjected to streptavidin for 15 hrs. To estimate the RBC lysis the absorbance of supernatant at 410 nm was measured post centrifugation at 1000 g to remove RBCs and debris. Optical density (OD) at 410 nm was plotted on Y-axis as representation of RBC lysis. Cond-A-Sulfo-NHS-Biotin; Cond-B-Sulfo-NHS-SS-Biotin; Cond-C-Sulfo-NHS-LC-Biotin; Cond-D-Sulfo-NHS-LC-LC-Biotin and Cond-E-Sulfo-NHS-PEG4-Biotin. OD of supernatants from streptavidin untreated and treated biotinylated RBCs at 15 hrs is represented with white bars, black bars respectively and OD of unbiotinylated RBCs with SA is shown with black bar. Similar levels of RBC lysis were observed with and without streptavidin in sulfo-LC-LC-biotinylated RBCs (Condition D). Following 15 hr of incubation increased in spontaneous RBC lysis was observed in conditions B and C without treatment with streptavidin as compared to un-biotinylated RBCs. However, compared to other conditions the lysis of RBCs was less in condition-E with and without streptavidin treatment.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “target cell directed moiety” refers to a moiety that interacts with a receptor on a target cell. The target cell directed moiety can be an entire molecule or a portion of a molecule. The target cell directed moiety can be included within or attached to another molecule as long as the target cell directed moiety is capable of interacting with the receptor on the target cell.

The term “target cell receptor” refers to a molecule on the surface of a target cell that, upon interaction with a target cell directed moiety/RBC complex, participates in and/or contributes to the stimulation a of biological effect in the target cell.

The term “red blood cells” includes hemoglobin-containing erythrocytes, erythroblasts and reticulocytes, as well as hemoglobin-depleted red blood cell “ghosts.”

The term “T cells” refers to CD4-positive or CD8-positive lymphocytes that express the CD3 antigen.

The term “activated T cells” refers to T cells that have undergone differentiation to a particular subset of T cell. Activated T cells include, but are not limited to, Th1 and Th2 subsets. Activated T cells include any T cell subtype and are not limited to any particular defined cytokine profile. As used herein, activated T cells may refer to either polyclonal or monoclonal populations of T cells.

The term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (VH and VL, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

The term “streptavidin” refers to a streptavidin protein and any fragment or derivation of a streptavidin protein which retains strong binding to biotin.

The term “biotin” refers to a biotin protein or any fragment or derivative of biotin which retains strong binding to avidin and streptavidin.

### Controlled Delivery

Disclosed herein is a method for controlled delivery of an agent in a subject that involves administering to the subject a biotinylated cell complex loaded with the agent; and then administering to the subject an effective amount of streptavidin crosslinking agent to rupture the biotinylated complex.

### Biotinylated Cell Complexes

In some embodiments, the biotinylated cell complex is a non-nucleated or enucleated cell. For example, the cell can be a red blood cell (RBC) or platelet. The use of complexes comprising RBCs for stimulation of a biological effect in target cells is disclosed in WO2004087876, which is incorporated by reference herein for the teaching of these complexes.

The use of an RBC or platelet as a component of a target cell stimulating complex offers distinct benefits for and advantages to stimulating a target cell.

Presentation of a target cell directed moiety on the surface of an RBC or platelet generally provides a local concentration of the moiety to the target cell through the presence of a number of moieties on the surface of an RBC or platelet. In some instances, coupling of the target cell directed moiety to the surface of an RBC or platelet allows for some mobility of the moiety when interacting with the target cell receptor and, accordingly, for mobility and/or aggregation of the target cell receptor. For some cell receptors, the ability to aggregate and/or move on the cell surface is important for effective cell signaling. In some instances, the RBC or platelet complexes can also be used to deliver agents (e.g., drugs, antigens, cytokines, chemokines, hormones) to particular cells and/or tissues. In addition, the use of RBC or platelet in the presentation of target cell directed moieties to target cells provide a source of oxygen to the cells in culture or in the individuals to which the complexes are administered.

Methods and techniques for attaching biotin to molecules and cells are well known in the art. See, for example, O'Shannessey et al. (1984) Immunol. Lett. 8:273-277; O'Shannessy et al. (1985) J. Appl. Biochem. 7:347-355; Wade et al. (1985) Biochem. J. 229:785-790; Rosenberg et al. (1986) J. Neurochem. 46:641-648 O'Shannessey et al. (1987) Anal. Biochem. 163:204-209; O'Shannessey et al. (1987) J Immunol. Meth. 99:153-161; Reisfield et al. (1987) Biochem. Biophys. Res. Com. 142:519-526; Bayer et al. (1988) Anal. Biochem. 170:271-281; Green (1965). Biochem. J. 94:23c-24c; Green (1975) Avidin. Adv. Protein. Chemistry, Academic Press, New York (Anfinsen et al. eds.) 29:85-133.

### Drugs for Delivery

The agents for controlled deliver can include one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin).

The agents for controlled deliver can include one or more of classes of steroids (e.g., Andranes (e.g., Testosterone).

The agents for controlled deliver can include one or more of classes of narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine).

The agents for controlled deliver can include one or more of classes of anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momiflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium).

The agents for controlled deliver can include one or more of classes of anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer (antineoplastic) drugs are available for combination with the present method and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Flurocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-I a, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

Other anti-neoplastic compounds include: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, atrsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorines, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docosanol, dolasetron, doxifluridine, droloxifene, dronabinol, duocannycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4-irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance genie inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfmosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene dichloride, topotecan, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer.

The agents for controlled deliver can include one or more radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

### Pathogen Treatment

Also disclosed is a method for killing a drug resistant pathogen in a subject that involves administering to the subject biotinylated binding agents that specifically bind one or more antigens on the pathogen; and then administering to the subject an effective amount of streptavidin crosslinking agent to rupture the pathogen.

### Biotinylated Antibodies

As described herein, the disclosed complexes may include an antibody that binds a receptor on the target cell, or the receptor binding portion of the antibody, as a target cell directed moiety. In such complexes, the anti-target cell antibody is coupled, either directed or indirectly, to the surface of disclosed cell-targeting complexes as described herein. As an example, the antibody can be labeled with biotin and coupled to the surface of a disclosed cell-targeting complexes through a biotin-avidin coupling.

Antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)₂) and recombinantly produced binding partners, single domain antibodies, hybrid antibodies, chimeric antibodies, single-chain antibodies, human antibodies, humanized antibodies, and the like. Generally, antibodies are understood to be reactive against a selected antigen on the surface of a cell if they bind with an affinity (association constant) of greater than or equal to 10⁻⁶ M.

Polyclonal antibodies against selected antigens on the surface of cells may be readily generated by one of ordinary skill in the art from a variety of warm-blooded animals such as—horses, cows, various fowl, rabbits, mice, or rats. In some cases, human polyclonal antibodies against selected antigens may be purified from human sources.

Monoclonal antibodies specific for selected antigens on the surface of cells may be readily generated using conventional techniques (see, for example, Harlow et al., 1988, supra, and U.S. Pat. Nos. RE 32,011, 4,902,614, 4,543,439, and 4,411,993). Hybridoma cells can be screened immunochemically for production of antibodies specifically reactive with an antigen, and monoclonal antibodies can be isolated. Other techniques may also be utilized to construct monoclonal antibodies (see, for example, Huse et al. (1989) Science 246:1275-1281; Sastry et al. (1989) Proc. Natl. Acad. Sci. USA 86:5728-5732; Alting-Mees et al. (1990) Strategies in Molecular Biology 3:1-9).

Similarly, binding partners may be constructed utilizing recombinant DNA techniques. For example, the genes which encode the variable region from a hybridoma producing a monoclonal antibody of interest are amplified using nucleotide primers for the variable region. These primers may be synthesized by one of ordinary skill in the art, or may be purchased from commercially available sources. The primers may be utilized to amplify-heavy or light chain variable regions, which may then be inserted into appropriate expression vectors. These vectors may then be introduced into cells, for example E. coli cells, for expression. Utilizing these techniques, large amounts of a single-chain protein containing a fusion of the H and V domains may be produced (see, for example, Bird et al. (1988) Science 242:423-426). In addition, such techniques may be utilized to change a “murine” antibody to a “human” antibody, without altering the binding specificity of the antibody.

As used herein, a “single domain antibody” (dAb) is an antibody which is comprised of a V_{H} domain, which reacts immunologically with a designated antigen. A dAb does not contain a domain, but may contain other antigen binding domains known to exist in antibodies, for example, the kappa and lambda domains. Methods for preparing dAbs are known in the art. See, for example, Ward et al. (1989) Nature 341:544-546. Antibodies may also be comprised of VH and V_{L} domains, as well as other known antigen binding domains. Examples of these types of antibodies and methods for their preparation are known in the art (see, e.g., U.S. Pat. No. 4,816,467).

Further exemplary antibodies include “univalent antibodies”, which are aggregates comprised of a heavy chain/light chain dimer bound to the Fc (i.e., constant) region of a second heavy chain. This type of antibody generally escapes antigenic modulation. See, e.g., Glennie et al. (1982) Nature 295:712-714.

Antibodies can be fragmented using conventional techniques and the fragments (including “Fab” fragments) screened for utility in the same manner as described above for whole antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Methods of producing Fab fragments of antibodies are known within the art and include, for example, proteolysis, and synthesis by recombinant techniques. For example, F(ab′)₂ fragments can be generated by treating antibody with pepsin. The resulting F(ab′)₂ fragment can be treated to reduce disulfide bridges to produce Fab′ fragments. “Fab” antibodies may be divided into subsets analogous to those described herein, i.e., “hybrid Fab”, “chimeric Fab”, and “altered Fab”.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth herein.

“Altered antibodies” are antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics. Changes in the variable region may be made to alter antigen binding characteristics. The antibody may also be engineered to aid the specific delivery of a molecule or substance to a specific cell or tissue site. The desired alterations may be made by known techniques in molecular biology, e.g., recombinant techniques, site directed mutagenesis, and other techniques.

By “humanized” is meant alteration of the amino acid sequence of an antibody so that fewer antibodies and/or immune responses are elicited against the humanized antibody when it is administered to a human. For the use of the antibody in a mammal other than a human, an antibody may be converted to that species format.

“Chimeric antibodies”, are antibodies in which the heavy and/or light chains are fusion proteins. Typically the constant domain of the chains is from one particular species and/or class, and the variable domains are from a different species and/or class. The invention includes chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al. (1985) Proc. Natl. Acad. Sci. U.S.A. 81:6851; Takeda et al. (1985) Nature 314:452; U.S. Pat. Nos. 4,816,567 and 4,816,397; European Patent Publications EP171496 and EP173494; United Kingdom patent GB 2177096B.

Bispecific antibodies may contain a variable region of an anti-target cell receptor antibody and a variable region specific for at least one antigen on the surface of an RBC or platelet. In other cases, bispecific antibodies may contain a variable region of an anti-target cell receptor antibody and a variable region specific for a linker molecule. In other cases, bispecific antibodies may contain a variable region specific for at least one antigen on the surface of an RBC or platelet and a variable region specific for a linker molecule. Bispecific antibodies may be obtained forming hybrid hybridomas, for example by somatic hybridization. Hybrid hybridomas may be prepared using the procedures known in the art such as those disclosed in Staerz et al. (1986, Proc. Natl. Acad. Sci. U.S.A. 83:1453) and Staerz et al. (1986, Immunology Today 7:241). Somatic hybridization includes fusion of two established hybridomas generating a quadroma (Milstein et al. (1983) Nature 305:537-540) or fusion of one established hybridoma with lymphocytes derived from a mouse immunized with a second antigen generating a trioma (Nolan et al. (1990) Biochem. Biophys. Ada 1040:1-11). Hybrid hybridomas are selected by making each hybridoma cell line resistant to a specific drug-resistant marker (De Lau et al. (1989) J. Immunol. Methods 117:1-8), or by labeling each hybridoma with a different fluorochrome and sorting out the heterofluorescent cells (Karawajew et al. (1987) J Immunol. Methods 96:265-270).

Bispecific antibodies may also be constructed by chemical means using procedures such as those described by Staerz et al. (1985) Nature 314:628 and Perez et al. (1985) Nature 316:354. Chemical conjugation may be based, for example, on the use of homo- and heterobifunctional reagents with E-amino groups or hinge region thiol groups. Homobifunctional reagents such as 5,5′-dithiobis(2-nitrobenzoic acid) (DNTB) generate disulfide bonds between, the two Fabs, and O-phenylenedimaleimide (O-PDM) generate thioether bonds between the two Fabs (Brenner et al. (1985) Cell 40:183-190, Glennie et al. (1987) J Immunol. 139:2367-2375). Heterobifunctional reagents such as N-succinimidyl-3-(2-pyridylditio) propionate (SPDP) combine exposed amino groups of antibodies and Fab fragments, regardless of class or isotype (Van Dijk et al. (1989) Int. J. Cancer 44:738-743).

Bifunctional antibodies may also be prepared by genetic engineering techniques. Genetic engineering involves the use of recombinant DNA based technology to ligate sequences of DNA encoding specific fragments of antibodies into plasmids, and expressing the recombinant protein. Bispecific antibodies can also be made as a single covalent structure by combining two single chains Fv (scFv) fragments using linkers (Winter et al. (1991) Nature 349:293-299); as leucine, zippers coexpressing sequences derived from the transcription factors fos and jun (Kostelny et al. (1992) J. Immunol. 148:1547-1553); as helix-turn-helix coexpressing an interaction domain of p53 (Rheinnecker et al. (1996) J. Immunol. 157:2989-2997), or as diabodies (Holliger et al. (1993) Proc. Natl. Acad. Sci. U.S.A. 90-: 6444-6448).

A tetrameric immunological complex may be prepared by mixing a first monoclonal antibody which is capable of binding to at least one receptor on the surface of a target cell and a second monoclonal antibody which is capable of binding to a moiety on an RBC or platelet. The first and second monoclonal antibodies are from a first animal species. The first and second antibodies are reacted with monoclonal antibodies of a second animal species which are directed against the Fc-fragments of the antibodies of the first animal species. The first and second antibody may also be reacted with the F(ab′)₂ fragments of monoclonal antibodies of a second animal species which are directed against the Fc-fragments of the antibodies of the first animal species. See, for example, U.S. Pat. No. 4,868,109. For example, the first and second antibody may be reacted with an about equimolar amounts of the monoclonal antibodies of the second animal species or of the F(ab′)₂ fragments thereof.

### Pathogens

In some embodiments, the disclosed methods can be used to target any cell infected with a pathogen that causes an antigen to be expressed on the infected cell for which a binding agent is available and/or can be produced. In some embodiments, the disclosed methods can be used to target the long-lasting reservoir of the viral or other pathogen infected cells dormant virus, such as HIV, HSV, etc., Chickenpox, Common cold, Diphtheria, E. coli, Giardiasis, HIV/AIDS, Infectious mononucleosis, Influenza (flu), Lyme disease, Malaria, Measles, Meningitis, Mumps, Poliomyelitis (polio), Pneumonia, Rocky mountain spotted fever, Rubella (German measles), Salmonella infections, Severe acute respiratory syndrome (SARS), Sexually transmitted diseases, Shingles (herpes zoster), Tetanus, Toxic shock syndrome, Tuberculosis, Viral hepatitis, West Nile virus, Whooping cough (pertussis), Acute Flaccid Myelitis (AFM), Anaplasmosis, Anthrax, Babesiosis, Botulism, Brucellosis, Campylobacteriosis, Carbapenem-resistant Infection (CRE/CRPA), Chancroid, Chikungunya Virus Infection (Chikungunya), Chlamydia, Ciguatera (Harmful Algae Blooms (HABs)), Clostridium Difficile Infection, Clostridium Perfringens (Epsilon Toxin), Coccidioidomycosis fungal infection (Valley fever), COVID-19 (Coronavirus Disease 2019), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E. coli infection, Shiga toxin-producing (STEC), Eastern Equine Encephalitis (EEE), Ebola Hemorrhagic Fever (Ebola), Ehrlichiosis, Encephalitis, Arboviral or parainfectious, Enterovirus Infection, Non-Polio (Non-Polio Enterovirus), Enterovirus Infection, D68 (EV-D68), Giardiasis (Giardia), Glanders, Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomavirus (HPV), Influenza (Flu), Lead Poisoning, Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis, Viral (Meningitis, viral), Meningococcal Disease, Bacterial (Meningitis, bacterial), Middle East Respiratory Syndrome Coronavirus (MERS-COV), Multisystem Inflammatory Syndrome in Children (MIS-C), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis (Parrot Fever), Pthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scabies Infestation (Scabies), Scombroid, Septic Shock (Sepsis), Severe Acute Respiratory Syndrome (SARS), Shigellosis gastroenteritis (Shigella), Smallpox, Methicillin-resistant Staphyloccal Infection (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A (invasive)), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichomoniasis (Trichomonas infection), Trichonosis Infection (Trichinosis), Tuberculosis (TB), Tuberculosis (Latent) (LTBI), Tularemia (Rabbit fever), Typhoid Fever, Group D, Typhus, Vaginosis, bacterial (Yeast Infection), Vaping-Associated Lung Injury (e-Cigarette Associated Lung Injury), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), and Zika Virus Infection (Zika).

### Binding Agents

In some embodiments, the biotinylated complex is coated with one or more binding agents for targeted delivery. Examples of binding agents include antibodies, ligands, receptors, aptamers, DNA, RNA, nucleic acids and PNA. Other suitable binding agents suitable for targeted delivery in vivo can also be adapted for use in the disclosed methods.

In some embodiments, the biotinylated complex causes physical strain on the membrane of a pathogen to rupture it. For example, strain can be induced by targeting surface protein, lipid, glycans, carbohydrates etc. with a binding agent and then crosslinking the binding agents with a crosslinking agent.

Tables 1 and 2 provide examples of pathogen targets for use in the disclosed compositions and methods.

TABLE 1

| Transmembrane | | |
|---|---|---|
| OMP | | |
| (Accession | | |
| number) | Species | Signal peptide |
| Msp | T. | MKKILAILMILVLVG |
| (JC6005) | denticola | GVAFA |
| | | (SEQ ID NO: 2) |
| OmpL1 | L. | MIRNISKALLILAVA |
| (AAA74591) | kirschneri | LSSAASLSA |
| | | (SEQ ID NO: 3) |
| Oms28 | B. | MTKIFSNLIINGLLF |
| (AAC66258) | burgdorferi | GFVSLNVFA |
| | | (SEQ ID NO: 4) |
| Oms66/P66 | B. | MKSHILYKLIIFLTT |
| (AAC66949) | burgdorferi | SAAIFA |
| | | (SEQ ID NO: 5) |
| p13 | B. | MNKLLIFVLATFCVF |
| (AAD28360) | burgdorferi | SSFAQA |
| | | (SEQ ID NO: 6) |
| TROMP1 | T. | MHQNSPKQCHLIRER |
| (AAA92353) | pallidum | ICACVLALGMLTGFH |
| | | AFGSKDAAA |
| | | (SEQ ID NO: 7) |
| DbpA | B. | MIKCNNKTFNNLLKL |
| (NP_045697) | burgdorferi | TILVNLLISC |
| | | (SEQ ID NO: 8) |
| LipL21 | L. | MINRLIALSLATMIF |
| (AAO88274) | kirschneri | AAC |
| | | (SEQ ID NO: 9) |
| LipL32 | L. | MKKLSILAISVALFA |
| (AAF60198) | kirschneri | SITAC |
| | | (SEQ ID NO: 10) |
| LipL36 | L. | MRRNIMKIAAVAALT |
| (AAC12923) | kirschneri | VALTAC |
| | | (SEQ ID NO: 11) |
| LipL41 | L. | MRKLSSLISVLVLLM |
| (AAB06799) | kirschneri | FLGNC |
| | | (SEQ ID NO: 12) |
| OspA | B. | MKKYLLGIGLILALI |
| (AAC66260) | burgdorferi | AC |
| | | (SEQ ID NO: 12) |
| OspB | B. | MRLLIGFALALALIG |
| (AAC66243) | burgdorferi | C |
| | | (SEQ ID NO: 13) |
| OspC | B. | MKKNTLSAILMTLFL |
| (AAC66329) | burgdorferi | FISC |
| | | (SEQ ID NO: 14) |
| OspE | B. | MNKKMKMFIVYAVFI |
| (AAB63439) | burgdorferi | LIGAC |
| | | (SEQ ID NO: 15) |
| SmpA | B. | MNKKIFTLFLVVAAS |
| (S33585) | hyodysenteriae | AIFAVSC |
| | | (SEQ ID NO: 16) |

TABLE 2
List of genes encoding membrane proteins

| | | | | | | | | |
|---|---|---|---|---|---|---|---|---|
| acrZ | envY | mdtQ | pgaA | yaiW | ynfB | amiD | fadL | mepS |
| bamA* | fepA | mlaA | phoE | yceK | yqhH | bamB | fhuA | mliC |
| bamD* | fimD | mltB | pqiC | yddL* | yzcX | bamE | fiu | mltC |
| mltF | rhsB | yehB | bhsA | flu | nanC | rhsD | yfaL | blc |
| btuB | gspD | nlpE | rzoD | yfeN | chiP | hofQ | nlpl | rzoR |
| skp | yghG | csgE | loiP | ompF | slp | ygiB | csgF | lolB |
| lpp | ompN | tamB | yiaT | ecnA | mdtP | ompT | tolC | yjbF |
| yjgL | elfC | lptD* | pagP | wza | ylil | emtA | lptE* | pal |
| pgaB | ybgQ | ypjA | appX | fecA | mipA | pgpB | ybhC | ypjB |
| pldA | ychO | yraJ | bamC | fhuE | mitA | ppk | yddB | yraP |
| qseG | ydeT | bcsC | flgG | mltD | rcsF | ydiY | bglH | flgH |
| gfcD | nfrA | rlpA | yfaZ | borD | gfcE | nlpD | rsxG | yfcU |
| yfgH | cirA | htrE | ompA | sfmD | yfiB | csgB | lamB | ompC |
| ompL | slyB | yhcD | csgG | lpoA | ompG | tamA | yiaD* | cusC |
| ecnB | lpoB | ompW | tsx | yjbH | ecpC | lptA* | ompX | uidC |
| yaiO | yncD | | | | | | | |

### Streptavidin Crosslinking Agent

Disclosed herein for use in the disclosed methods are streptavidin crosslinking agents that can bind and crosslink biotin molecules on the surface of a cell.

The streptavidin can be wild-type streptavidin, a streptavidin mutein, or a streptavidin-like polypeptide capable of binding and crosslinking biotin molecules. Wild-type streptavidin (wt-streptavidin), the amino acid sequence disclosed by Argarana et al., Nucleic Acids Res. 14 (1986) 1871-1882 is referred to.

In some embodiments, wild-type streptavidin (wt-streptavidin) has the amino acid sequence

(SEQ ID NO: 1)
MRKIVVAAIAVSLTTVSITASASADPSKDSKAQVSAAEAGITGTWYNQL
GSTFIVTAGADGALTGTYESAVGNAESRYVLTGRYDSAPATDGSGTALG
WTVAWKNNYRNAHSATTWSGQYVGGAEARINTQWLLTSGTTEANAWKST
LVGHDTFTKVKPSAASIDAAKKAGVNNGNPLDAVQQ.

Streptavidin muteins are polypeptides which are distinguished from the sequence of wild-type streptavidin by one or more amino acid substitutions, deletions or additions and which retain the binding properties of wt-streptavidin. Streptavidin-like polypeptides and streptavidin muteins are polypeptides which essentially are immunologically equivalent to wild-type streptavidin and are in particular capable of binding biotin, biotin derivative or biotin analogues with the same or different affinity as wt-streptavidin. Streptavidin-like polypeptides or streptavidin muteins may contain amino acids which are not part of wild-type streptavidin or they may include only a part of wild-type streptavidin. Streptavidin-like polypeptides are also polypeptides which are not identical to wild-type streptavidin, since the host does not have the enzymes which are required in order to transform the host-produced polypeptide into the structure of wild-type streptavidin.

The term streptavidin also includes streptavidin tetramers and streptavidin dimers, in particular streptavidin homotetramers, streptavidin homodimers, streptavidin heterotetramers and strepavidin heterodimers. Each subunit normally has a binding site for biotin or biotin analogues or for streptavidin-binding peptides.

Examples of streptavidins or streptavidin are mentioned, for example, in U.S. Pat. Nos. 10,844,099, 6,022,951, WO1986/002077, DE19641876A1, WO1998/040396, and WO1996/024506, which are incorporated by reference for the teaching of these streptavidins and streptavidin (avidin, neutravidin).

### Formulations and Routes of Administration

In some embodiments, the disclosed complexes are used in the preparation of medicaments, for treating the conditions described herein. These complexes are administered as pharmaceutically acceptable compositions. The complexes may be administered by any suitable means, including, but not limited to, intravenously, parenterally or locally. The complexes can be administered in a single dose by bolus injection or continuous infusion or in several doses over selected time intervals in order to titrate the dose.

The particular administration mode selected will depend upon the particular composition, treatment, cells involved, etc., The volume will depend upon, for example, the type of cell administered, the disorder treated and the route of administration.

As used herein, “pharmaceutically acceptable excipient” includes any material which, when combined with an active ingredient of a composition, allows the ingredient to retain biological activity and without causing disruptive reactions with the subject's immune system. Various pharmaceutically acceptable excipients are well known in the art.

Exemplary pharmaceutically acceptable excipients include sterile aqueous or non-aqueous solutions and suspensions. Examples include, but are not limited to, any of the standard pharmaceutical excipients such as a phosphate buffered saline solution, water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Compositions comprising such excipients are formulated by well known conventional methods (see: for example, Remington's Pharmaceutical Sciences, 18th Ed., Mack Publishing Co.).

### SPECIFIC EMBODIMENTS

Embodiment 1. A method for controlled delivery of an agent in a subject, comprising
administering to the subject a biotinylated cell complex loaded with the agent; and
administering to the subject an effective amount of streptavidin crosslinking agent to rupture the biotinylated complex,
wherein the biotinylated cell complex is a non-nucleated or enucleated cell or a liposome.

Embodiment 2. The method of embodiment 1, wherein the cell is a red blood cell (RBC) or platelet or liposome.

Embodiment 3. The method of embodiment 1 or 2, wherein the biotinylated complex is coated with one or more binding agents for targeted delivery.

Embodiment 4. The method of embodiment 3, wherein the binding agent comprises an antibody.

Embodiment 5. The method of embodiment 3, wherein the binding agent comprises a ligand or receptor.

Embodiment 6. The method of embodiment 3, wherein the binding agent comprises a peptide, lipid, carbohydrate, oligonucleotide, or PNA conjugated with biotin.

Embodiment 7. The method of any one of embodiments 3 to 6, wherein the binding agent specifically binds a tumor antigen.

Embodiment 8. The method of embodiment 7, wherein the agent comprises costimulatory molecules, death receptors, chemokines, and/or cytokines configured to kill cancer cells.

Embodiment 9. A method for killing a drug resistant pathogen in a subject, comprising
administering to the subject biotinylated binding agents that specifically bind one or more antigens on the pathogen; and
administering to the subject an effective amount of streptavidin crosslinking agent to rupture the pathogen.

Embodiment 10. The method of embodiment 9, wherein the binding agent comprises an antibody.

Embodiment 11. The method of embodiment 9, wherein the binding agent comprises a ligand or receptor.

Embodiment 12. The method of embodiment 9, wherein the binding agent comprises an aptamer.

Embodiment 13. The method of embodiment 9, wherein the pathogen is drug resistant

Embodiment 14. The method of any one of embodiments 9 to 13, wherein the target is a host cell infected with a plasmodium parasite.

Embodiment 15. The method of embodiment 14, wherein the antigen is selected from the group consisting of Merozoite Surface Protein 1 (MSP1), MSP2, MSP3, MSP4, MSP5, MSP6, MSP7, MSP8, MSP9, and MSP10.

Embodiment 16. The method of any one of embodiments 9 to 13, wherein the target is a host cell infected with a HIV-1 or HIV-2.

Embodiment 17. The method of embodiment 16, wherein the antigen is selected from the group consisting of Gp120, Gp160, gP41, VPU, and Nef.

Embodiment 18. The method of any one of embodiments 9 to 13, wherein the target is a host cell infected with hepatitis B virus (HBV).

Embodiment 1. The method of embodiment 18, wherein the antigen is selected from the group consisting of HBV-M protein, HBV-L protein, and HBV-S protein.

Embodiment 19. The method of any one of embodiments 9 to 13, wherein the target is a host cell infected with hepatitis C virus (HCV).

Embodiment 20. The method of embodiment 20, wherein the antigen is HCV E1 glycoprotein or HCV E2 glycoprotein.

Embodiment 21. The method of any one of embodiments 9 to 13, wherein the target is a host cell infected with SARS-Cov-2 virus.

Embodiment 22. The method of embodiment 21, wherein the antigen is selected from the group consisting of spike(S), nucleocapsid (N), envelop (E), and membrane (M) proteins.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Procedure for Biotinylation of RBCs and to Evaluate Effect of Streptavidin on Biotinylated-RBCs (B-RBCs)

In this example biotinylation of RBCs was performed using amine reactive biotinylation agents. Amines, lysine E-amines and N-terminal α-amines, are the most abundant groups in protein molecules and represent the most common target for biotinylation. Amine reactive biotinylation reagents contain reactive group off biotin's valeric acid side chain that are able to form covalent bonds with primary amines present on the surface proteins. Several NHS-biotin derivatives available with different length spacer arm off the valeric acid side chain, terminating in NHS-ester. In our examples we have evaluated five different NHS-X-Biotins, whereas X represent the spacer arm. We have tested water soluble sulfo derivatives of NHS-Biotin, NHS-SS-Biotin, NHS-LC-Biotin, NHS-LC-LC-Biotin and NHS-PEG4-Biotin. Of these the NHS-Biotin is the simplest biotinylation reagent available, the valeric acid carboxylate of D-biotin activated to an NHS ester for direct modification of amine groups in proteins with shortest spacer arm off the valeric acid side chain whereas PEG-4 has the long spacer arm as compared to other tested. RBCs are labeled with Sulfo-NHS-X-Biotin, an amine reactive biotinylation reagent that is soluble in water, but impermeable to plasma membranes as per following protocol.

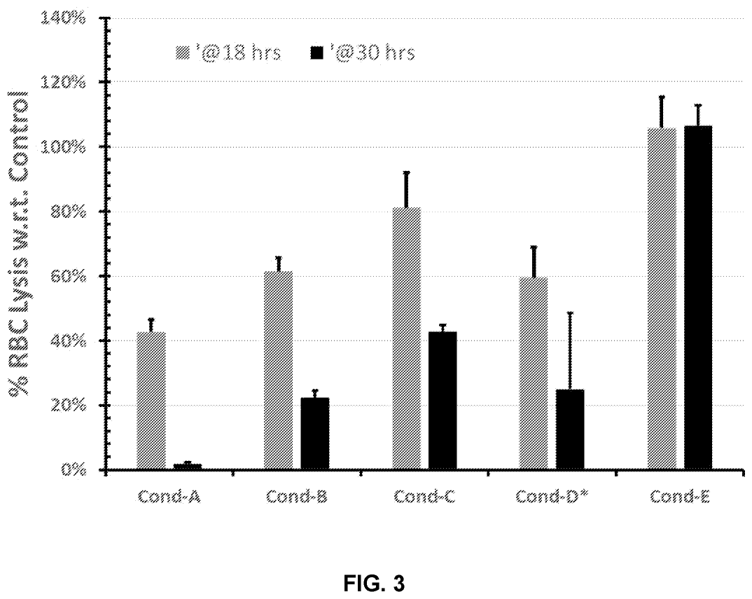

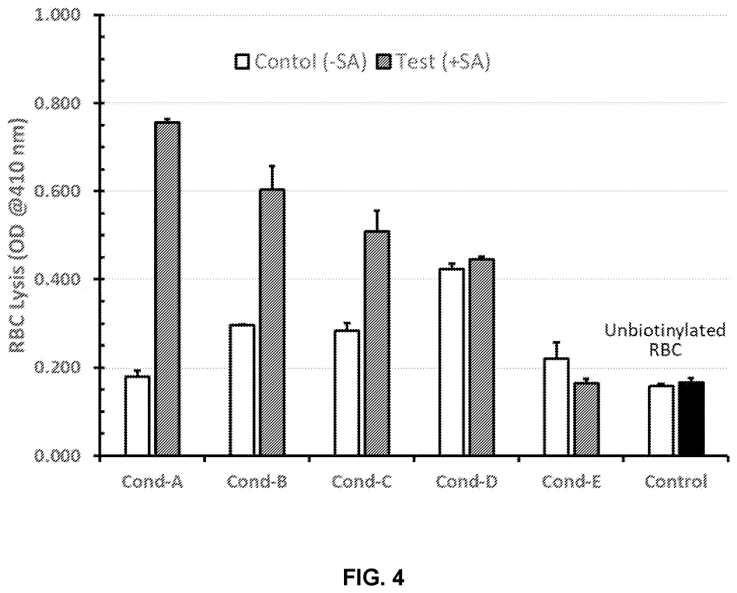

RBCs were washed and suspended in PBS buffer at 100 million per ml and freshly prepared NHS-X-Biotin reagent is added at the final concentration of 0.1-20 mM Biotin. Reaction mixture is incubated at room temperature on continuous shaker for 30 min to 3 hrs. Following incubation unreactive excess biotin reagent was quenched by three washes with 100 mM glycine prepared in PBS. After the glycine wash Biotinylated-RBCs (B-RBCs) were further washed with RBC storage buffer and then used to evaluate effect of streptavidin. To evaluate effect of streptavidin on B-RBCs, 2×10⁶ B-RBCs per ml were incubated with 0.1 to 5 UM of SA at room temperature over 30 hrs time period. B-RBCs counts were performed at 15 hrs and 30 hrs using hematocytometer manual cell counting procedure (FIG. 3). In addition to evaluate the lysis of RBCs reaction mixture was centrifuge to remove the debris and intact RBCs and the optical density of supernatant was measure at absorbance max of hemoglobin i.e. 410 nm (FIG. 4).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for controlled delivery of an agent in a subject, comprising
(a) administering to the subject a biotinylated cell complex loaded with the agent; and
(b) administering to the subject an effective amount of streptavidin crosslinking agent to rupture the biotinylated complex,
wherein the biotinylated cell complex is a non-nucleated or enucleated cell or a liposome.

2. The method of claim 1, wherein the cell is a red blood cell (RBC) or platelet or liposome.

3. The method of claim 1, wherein the biotinylated complex is coated with one or more binding agents for targeted delivery.

4. The method of claim 3, wherein the binding agent comprises an antibody.

5. The method of claim 3, wherein the binding agent comprises a ligand or receptor.

6. The method of claim 3, wherein the binding agent comprises a peptide, lipid, carbohydrate, oligonucleotide, or PNA conjugated with biotin.

7. The method of claim 3, wherein the binding agent specifically binds a tumor antigen.

8. The method of claim 7, wherein the agent comprises costimulatory molecules, death receptors, chemokines, and/or cytokines configured to kill cancer cells.

9. A method for killing a drug resistant pathogen in a subject, comprising
(a) administering to the subject biotinylated binding agents that specifically bind one or more antigens on the pathogen; and
(b) administering to the subject an effective amount of streptavidin crosslinking agent to rupture the pathogen.

10. The method of claim 9, wherein the binding agent comprises an antibody, ligand, receptor, or aptamer.

11. (canceled)

12. (canceled)

13. (canceled)

14. The method of claim 9, wherein the target is a host cell infected with a plasmodium parasite.

15. The method of claim 14, wherein the antigen is selected from the group consisting of Merozoite Surface Protein 1 (MSP1), MSP2, MSP3, MSP4, MSP5, MSP6, MSP7, MSP8, MSP9, and MSP10.

16. The method of claim 9, wherein the target is a host cell infected with a HIV-1 or HIV-2.

17. The method of claim 16, wherein the antigen is selected from the group consisting of Gp120, Gp160, gP41, VPU, and Nef.

18. The method of claim 9, wherein the target is a host cell infected with hepatitis B virus (HBV).

19. The method of claim 18, wherein the antigen is selected from the group consisting of HBV-M protein, HBV-L protein, and HBV-S protein.

20. The method of claim 9, wherein the target is a host cell infected with hepatitis C virus (HCV).

21. The method of claim 20, wherein the antigen is HCV E1 glycoprotein or HCV E2 glycoprotein.

22. The method of claim 9, wherein the target is a host cell infected with SARS-Cov-2 virus.

23. The method of claim 22, wherein the antigen is selected from the group consisting of spike(S), nucleocapsid (N), envelop (E), and membrane (M) proteins.


SEQUENCE LISTING (16 sequences)

SEQ ID NO: 1 - protein, 183 aa, Homo sapiens
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 20 aa, Treponema denticola
MKKILAILMILVLVGGVAFA

SEQ ID NO: 3 - protein, 24 aa, Leptospira kirschneri
MIRNISKALLILAVALSSAASLSA

SEQ ID NO: 4 - protein, 24 aa, Borrelia burgdorferi
MTKIFSNLIINGLLFGFVSLNVFA

SEQ ID NO: 5 - protein, 21 aa, Borrelia burgdorferi
MKSHILYKLIIFLTTSAAIFA

SEQ ID NO: 6 - protein, 21 aa, Borrelia burgdorferi
MNKLLIFVLATFCVFSSFAQA

SEQ ID NO: 7 - protein, 39 aa, Treponema pallidum
MHQNSPKQCHLIRERICACVLALGMLTGFHAFGSKDAAA

SEQ ID NO: 8 - protein, 25 aa, Borrelia burgdorferi
MIKCNNKTFNNLLKLTILVNLLISC

SEQ ID NO: 9 - protein, 18 aa, Leptospira kirschneri
MINRLIALSLATMIFAAC

SEQ ID NO: 10 - protein, 20 aa, Leptospira kirschneri
MKKLSILAISVALFASITAC

SEQ ID NO: 11 - protein, 21 aa, Leptospira kirschneri
MRRNIMKIAAVAALTVALTAC

SEQ ID NO: 12 - protein, 20 aa, Borrelia burgdorferi
MRKLSSLISVLVLLMFLGNC

SEQ ID NO: 13 - protein, 16 aa, Borrelia burgdorferi
MRLLIGFALALALIGC

SEQ ID NO: 14 - protein, 19 aa, Borrelia burgdorferi
MKKNTLSAILMTLFLFISC

SEQ ID NO: 15 - protein, 20 aa, Homo sapiens
MNKKMKMFIVYAVFILIGAC

SEQ ID NO: 16 - protein, 22 aa, Brachyspira hyodysenteriae
MNKKIFTLFLVVAASAIFAVSC